(19) United States
(12) Patent Application Publication
(10) Pub. No.: US 2012/0016394 A1
(43) Pub. Date: Jan. 19, 2012

(54) Method of Performing a Surgical Procedure

(76) Inventors: Frank BONADIO, Bray (IE); Michael Amirfarzad Kia, Grand Blanc, MI (US)

(21) Appl. No.: 13/029,467

(22) Filed: Feb. 17, 2011

Related U.S. Application Data
(60) Provisional application No. 61/305,744, filed on Feb. 18, 2010.
(60) Provisional application No. 61/387,757, filed on Sep. 29, 2010.

(51) Int. Cl. A61B 17/22

(52) U.S. Cl. 606/159

(57) ABSTRACT

A method for performing a cholecystectomy procedure comprises providing an access device having a plurality of ports for reception of instruments. An incision is made in the abdomen of a patient and the access device is mounted to provide access through the incision. A first grasper device is inserted through a first port of the access device, a second grasper device is inserted through a second port of the access device and a dissector is inserted through a third port of the access device. At least a first portion of the gall bladder is detached from surrounding tissue.

### CROSS REFERENCE TO RELATED APPLICATIONS

This application claims the benefit of U.S. Provisional Application No. 61/305,744 filed on Feb. 18, 2010 and U.S. Provisional Application No. 61/387,757 filed on Sep. 29, 2010, the entire contents of both of which are herein incorporated by reference.

### INTRODUCTION

This invention relates to a method of performing a surgical procedure. In one embodiment this invention relates to a method of performing a laparoscopic cholecystectomy procedure. The invention also relates to instrument access devices.

### STATEMENTS OF INVENTION

According to the invention there is provided a method for performing a cholecystectomy procedure comprising the steps of:
providing an access device having a plurality of ports for reception of instruments;
making an incision in the abdomen of a patient;
mounting the access device to provide access through the incision;
inserting a first grasper device through a first port of the access device;
inserting a second grasper device through a second port of the access device;
inserting a dissector through a third port of the access device; and
detaching at least a first portion of the gall bladder from surrounding tissue.

In one embodiment the method comprises moving the partially dissected gall bladder using at least one of the grasper devices, and detatching another portion of the gall bladder from surrounding tissue.

In one case the method comprises grasping the fundus of the gall bladder using the first grasper device.

In one case the method comprises grasping the infundibulum of the gall bladder using the second grasper device.

In one aspect the invention provides a method for performing a cholecystectomy procedure comprising the steps of:
providing an access device having a plurality of ports for reception of instruments;
making an incision in the abdomen of a patient;
mounting the access device to provide access through the incision;
inserting a first grasper device through a first port of the access device;
inserting a second grasper device through a second port of the access device;
inserting a dissector through a third port of the access device;
grasping the fundus of the gall bladder using the first grasper device;
grasping the infundibulum of the gall bladder using the second grasper device;
detaching at least a first portion of the gall bladder from surrounding tissue using the dissector;
moving the partially detached gall bladder using at least one of the graspers to expose another portion of tissue surrounding the gall bladder; and
detaching the further portion of the gall bladder from surrounding tissue using the dissector.

In one embodiment the method comprises the step of inserting a camera through a fourth port of the access device in advance of inserting one or both of the grasper devices.

In one case the method comprises:—
removing some of the instruments from the ports; and
inserting instruments through the ports, and dissecting another portion of the gall bladder.

The first retractor device may comprise a fundus grasper device.

The second retractor device may comprise an infandibulum grasper. The infandibulum grasper may have a curved shaft portion.

In one embodiment the method comprises holding the grasper devices separate from the dissector. The grasper devices may be held by a surgical assistant.

In one case the method comprises the step of creating the wound opening. The wound opening may be created by creating a skin incision, and subsequently forcing tissue apart.

In one case the wound opening is created using a Hasson cut-down incision.

In one embodiment the method comprises the step of inserting an instrument access device at least partially through the wound opening.

The instrument access device may be inserted at least partially through the wound opening using an introducer device. The method may comprise the step of inserting at least part of the instrument access device into the introducer device.

In one case the method comprises the step of inserting the introducer device at least partially through the wound opening. The method may comprise the step of ejecting at least part of the instrument access device from the introducer device within the wound interior. The method may comprise the step of removing the introducer device from the wound opening.

In one embodiment the method comprises the step of retracting the wound opening.

The method may comprise the step of insufflating the wound interior.

In one embodiment one or more body parts are removed through one or more ports of the instrument access device.

In one case the method comprises the step of detaching the access device from a retractor member of the instrument access device. The one or more body parts may be removed through the retractor member.

The device of the invention comprises at least one instrument seal to effect a seal around at least one instrument extended through the device, the instrument seal being configured to be arranged in sealing relationship to a body of a patient. The device may have a distal anchoring member for location within a wound interior. The device may also has a retractor member extending proximally from the distal anchoring member to retract laterally the sides of a wound opening. In one case the device comprises a first instrument seal or valve to effect a seal around a first instrument extended through the device, and a second instrument seal to effect a seal around a second instrument extended through the device. By providing the two seal arrangement, this ensures that insertion or manipulation or removal of the second instrument does not adversely effect the seal around the first instrument. The device may comprise a third instrument seal to effect a seal around a third instrument extended through the device. The first instrument seal may be spaced apart from the second instrument seal. The first instrument seal may be formed separately from the second instrument seal. The first instrument seal may have a larger radial dimension than the second instrument seal. The instrument seal may be of a gelatinous elastomeric material.

In one case the device comprises a proximal member for location externally of a wound opening. The retractor member may extend at least between the distal anchoring member and the proximal member. The retractor member may extend in two layers between the distal anchoring member and the proximal member. A first end portion of the retractor member may be fixed to the proximal member. The retractor member may be movable relative to the distal anchoring member. A second end portion of the retractor member may be movable relative to the proximal member. The retractor member may extend distally from the proximal member to the distal anchoring member, may be looped around the distal anchoring member, and may extend proximally from the distal anchoring member to the proximal member. The proximal member may comprise an inner part and an outer part. The retractor member may extend between the inner part and the outer part.

In another embodiment the instrument seal is spaced proximally of the proximal member. The device may comprise at least one connector member to connect the proximal member to the at least one instrument seal. The connector member facilitates a degree of lateral movement of the instrument while maintaining the seal. The connector member may comprise a sleeve. The connector member may be of a laterally flexible material. The connector member may be of a longitudinally rigid material. The connector member may be of a rubber-like material. The connector member may be of a longitudinally flexible material.

In another case the instrument seal is mounted to the connector member. The instrument seal may be releasably mounted to the connector member. The instrument seal may comprise a mounting part to mount the instrument seal to the connector member. The mounting part may be of a rigid material. The instrument seal may comprise a sealing part to effect a seal around an instrument extended through the device, the sealing part being overmoulded over at least part of the mounting part.

In one embodiment the connector member is mounted to the proximal member. The connector member may be releasably mounted to the proximal member. The connector member may be mounted to the proximal member in an interference fit arrangement. The connector member may be mounted to the proximal member in a snap-fit arrangement. The connector member may comprise at least one protrusion for engagement with the proximal member. The protrusion can be resilient. The device may comprise a clamp member to clamp the connector member to the proximal member. The connector member may be inclined relative to the proximal member. The device may comprise a reinforcement element to reinforce the connector member. The reinforcement element may be of a rigid material. The reinforcement element may be embedded within the connector member.

### BRIEF DESCRIPTION OF THE DRAWINGS

The invention will be more clearly understood from the following description of some embodiments thereof, given by way of example only, with reference to the accompanying drawings, in which:—

FIGS. 1 and 2 are views of an instrument access device according to the invention;

FIG. 3 is an exploded, isometric view of another instrument access device according to the invention;

FIG. 4 is a cross sectional view of the instrument access device of FIG. 4;

FIG. 5 is an isometric view of a pack;

FIG. 6 is an isometric view of a device suitable for use in performing a surgical procedure according to the invention;

FIG. 7 is an isometric view of another device for use in forming the produce;

FIGS. 8 to 15 are isometric views illustrating insertion of an instrument access device into a wound opening;

FIGS. 16 to 22 are isometric views illustrating various steps in performing a surgical procedure using surgical instruments inserted through an instrument access device;

FIGS. 23 to 29 are isometric views illustrating further steps in performing the surgical procedure;

FIGS. 30 to 37 illustrate steps in performing a surgical procedure using surgical instruments inserted through an instrument access device;

FIG. 38 is an exploded perspective view of another instrument device;

FIG. 39 is a perspective view of the device of FIG. 38 assembled;

FIG. 40 is a top plan view of the device of FIG. 39;

FIG. 41 is an elevational view from one side of the device of FIG. 39;

FIG. 42 is an elevational view from another side of the device of FIG. 39;

FIGS. 43 and 44 are isometric views of a valve assembly of an instrument access device;

FIG. 45 is an exploded isometric view of one valve assembly;

FIG. 46 is a cross sectional view of part of the valve assembly of FIG. 45;

FIG. 47 is an exploded isometric view of another valve assembly;

FIG. 48 is a cross sectional view of part of the valve assembly of FIG. 47;

FIG. 49 is an exploded isometric view of a further valve assembly; and

FIG. 50 is a cross sectional view of part of the valve assembly of FIG. 49.

### DETAILED DESCRIPTION

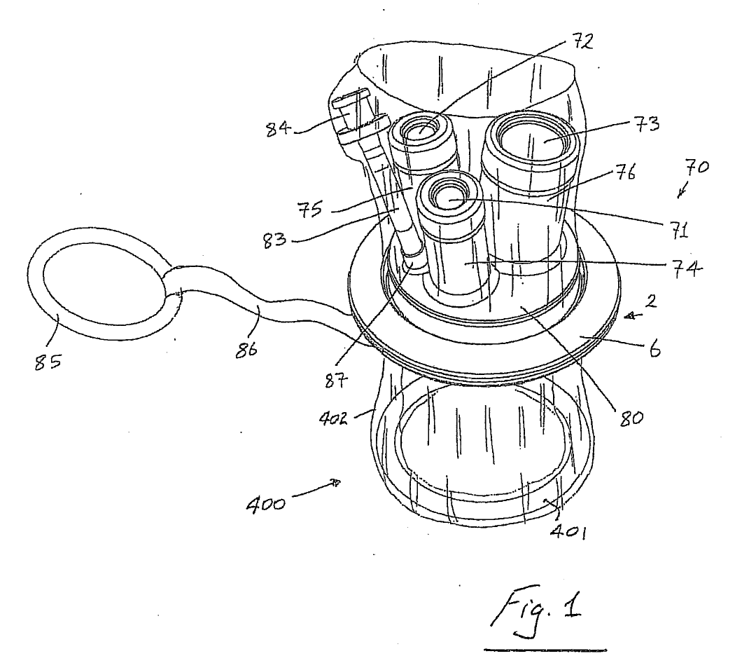

Referring initially to FIGS. 1 and 2 there is illustrated an instrument access device 70 is suitable for use during laparoscopic surgery to facilitate instrument access to an insufflated abdominal cavity while maintaining pneumoperitoneum. A distal anchoring ring is located within a wound interior, in use. In this case the distal anchoring ring is provided in the form of an O-ring 401. A proximal ring assembly 2 is located externally of a wound opening, in use. The proximal ring assembly 2 comprises an inner ring part 5 and an outer ring part 6. In this case the inner ring part 5 is provided in the form of an O-ring.

The instrument access device in this case comprises a retractor 400 and having a distal anchoring ring in the form of an O-ring 401. A retractor member comprises a sleeve 402 which in this case extends in two layers between the distal anchoring ring 401 and the proximal ring assembly 2. The retractor member 402 may be employed to retract laterally the sides of a wound opening. In this case the retractor member extends between the distal anchoring ring and the proximal ring assembly 2 in two layers. A first end of the retractor member is fixed to the inner ring part 5. The retractor member extends distally from the inner ring part 5 to the distal anchoring ring, is looped around the distal anchoring ring, extends proximally from the distal anchoring ring to the proximal ring assembly 2, and extends proximally between the inner ring part 5 and the outer ring part 6. The retractor member is slidably movable relative to the distal anchoring ring, and a second end of the retractor member is slidably movable between the inner ring part 5 and the outer ring part 6.

One such retractor is described in our US 2005-0090717 A, the entire contents of which are incorporated herein by reference.

In this case the device 70 comprises a first instrument seal 71, a second instrument seal 72, a third instrument seal 73, a first connector sleeve 74, a second connector sleeve 75, and a third connector sleeve 76.

Each instrument seal 71, 72, 73 may be employed to effect a seal around a separate instrument extended through the device 70. Each instrument seal 71, 72, 73 is formed separately from the other instrument seals 71, 72, 73, and is spaced apart from the other instrument seals 71, 72, 73. The first instrument seal 71 has a diameter equal to the diameter of the second instrument seal 72. The third instrument seal 73 has a larger diameter than the second instrument seal 72.

Each connector sleeve 74, 75, 76 connects the proximal ring assembly 2 to one of the instrument seals 71, 72, 73.

Each instrument seal 71, 72, 73 comprises a sealing part 77 and a mounting part 78 of a rigid material. The sealing part 77 effects a seal around an instrument extended through the device 70. The mounting part 78 facilitates releasable mounting of the instrument seal 71, 72, 73 to the connector sleeve 74, 75, 76 in a gas-tight manner. The mounting part 78 comprises an outwardly protruding barb 79 for an interference fit between the mounting part 78 and the connector sleeve 74, 75, 76. The sealing part 77 is overmoulded over part of the mounting part 78 to connect the sealing part 77 to the mounting part 78.

The device 70 comprises a connector base 80 to releasably mount the connector sleeves 74, 75, 76 to the inner ring part 5 in a gas-tight manner. The base 80 comprises outwardly protruding ridges 81 for an interference fit between the base 80 and the inner ring part 5.

A rigid reinforcement ring 82 is embedded within the base 80 to reinforce the base 80.

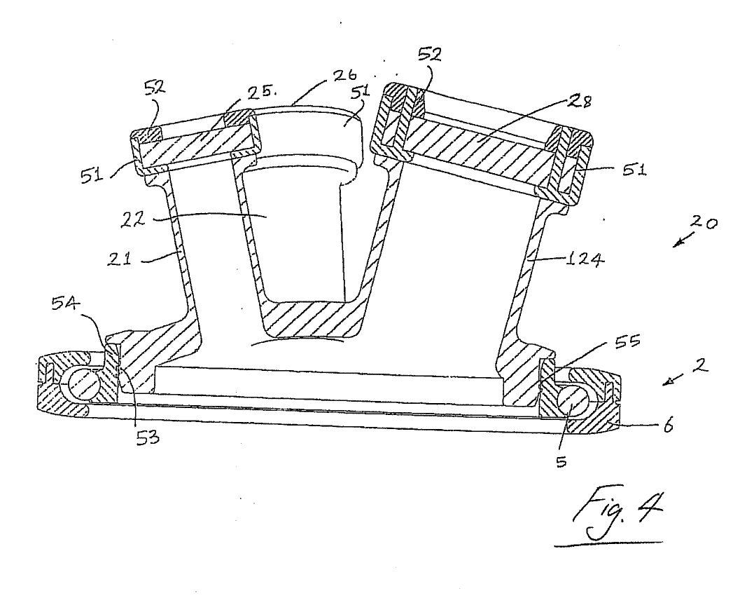

Each connector sleeve 74, 75, 76 is inclined relative to the proximal ring assembly 2 (FIG. 7).

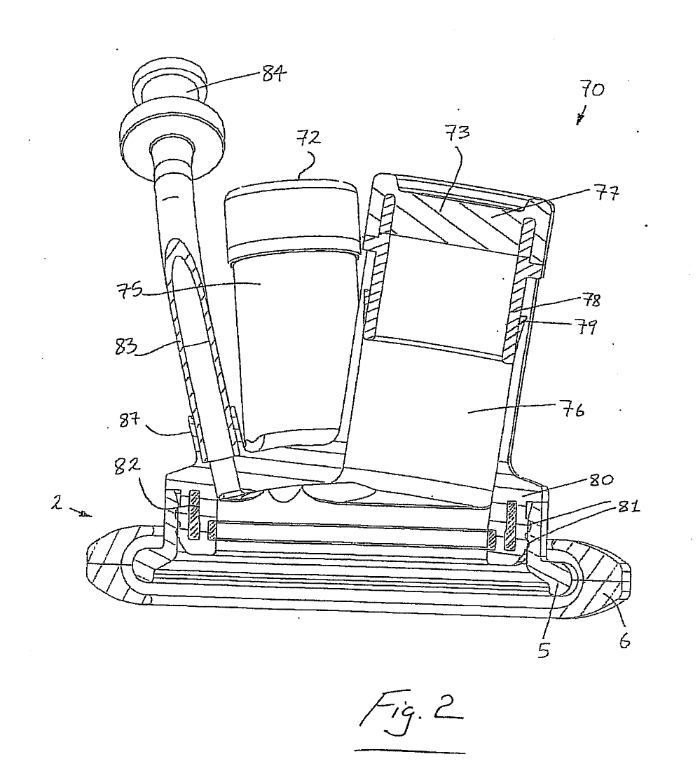

Referring to FIGS. 3 and 4 there is illustrated a further instrument access device 20 according to the invention, which is similar to the instrument access device of FIGS. 1 and 2, and similar elements are assigned the same reference numerals.

In this case the device 20 comprises a first instrument seal 25, a second instrument seal 26, a third instrument seal 27, a fourth instrument seal 28, a first connector sleeve 21, a second connector sleeve 22, a third connector sleeve 123, and a fourth connector sleeve 124.

Each instrument seal 25, 26, 27, 28 may be employed to effect a seal around a separate instrument extended through the device 20. Each instrument seal 25, 26, 27, 28 is formed separately from the other instrument seals 25, 26, 27, 28, and is spaced apart from the other instrument seals 25, 26, 27, 28. The first instrument seal 25 has a smaller diameter than the second instrument seal 26. The second instrument seal 26 has a diameter equal to the diameter of the third instrument seal 27. The fourth instrument seal 28 has a larger diameter than the third instrument seal 27.

Each connector sleeve 21, 22, 123, 124 connects the proximal ring assembly 2 to one of the instrument seals 25, 26, 27, 28.

FIG. 3 illustrates an elastomeric multiport cap 20 with for example a 5 mm port 25, a 10 mm port 26, a 10 mm port 27, a 12 mm port 28, and integrally moulded sealing flanges 53. Various combinations of ports are possible. FIG. 3 also illustrates the proximal ‘O’ ring 5, the outer proximal ring 6, and a docking ring 54 for the rubber multiport cap. The device 20 is suitable for large incisions, for example 2-4 cm.

FIG. 4 illustrates the seal 25, for example 5 mm instrument port, the seal 26, for example 10 mm instrument port, the seal 28, for example 12 mm instrument port, the caps 52, the housings 55, the docking ring 54 for the rubber port cap, the gas seal engagement point 55, the proximal ‘O’ ring 5, and the outer proximal ring 6.

FIGS. 3 and 4 illustrate the series of valves 25, 26, 27, 28 mounted on the elastomeric offset tubes 21, 22, 123, 124. The offset sleeves 21, 22, 123, 124 may be of rubber, which has enough lateral flexibility for full instrument range of motion, but the longitudinal structural rigidity of the tubes 21, 22, 123, 124 means the surgeon can introduce an instrument as a single-handed procedure.

Referring to FIGS. 43 to 50 the insertion devices typically comprise a lipseal 610 through which an instrument 611 is insertable and a second seal member 612 having a passageway extending therethrough, through which the instrument 611 is insertable. A first insertion device 602 also has a reducer cap 615 which has a further lipseal 616 which is smaller than the lipseal 610. To insert large diameter instruments, the cap 615 is removed. To insert smaller diameter instruments the cap 615 is in place.

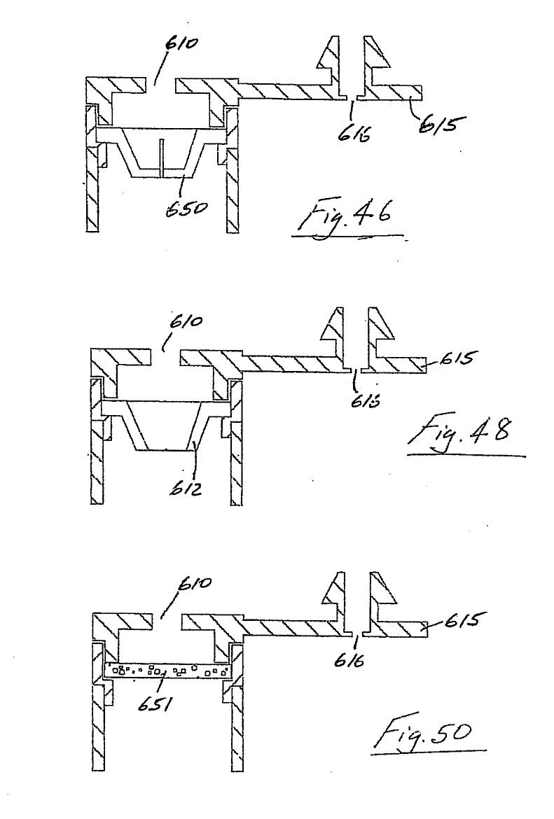

Referring to FIGS. 47 to 48 the second seal member 612 may comprise a duckbill valve through which the instrument 611 passes. The duckbill valve 612 provides sealing engagement with the instrument shaft whilst accommodating lateral movement of the instrument as illustrated. Alternatively, as illustrated in FIGS. 45 and 46 the second valve may comprise a multicusp valve such as a tricuspid valve 650. In another case as illustrated in FIGS. 49 and 50 the second valve may comprise a foam or gel.

The lipseal valve 610 is located proximally of the duckbill valve 612 so that a double seal is provided to substantially prevent leakage of insufflation gas.

The lipseal 610 may be of any suitable material. For example it may be of an elastomeric material, a foam—type material or a gelatinous material. The duckbill valve 612 may be of any suitable material. For example, it may be of a flexible polymeric material.

The second and third instrument insertion devices 603, 604 may be of the same or different construction as that of the first instrument insertion device 602.

The instrument access device of the invention is suitable for use during laparascopic surgery to facilitate instrument access to an insufflated abdominal cavity while maintaining pneumoperitoneum.

Referring to FIGS. 38 to 42 there is illustrated another instrument access device 700 which in this case comprises a first instrument insertion device 701, a second instrument insertion device 702, a third instrument insertion device 703, and a fourth instrument insertion device 704. The first instrument insertion device 701 can accommodate an instrument of up to 10 mm diameter and has a reducer cap 715 which, when closed, converts the insertion device to accommodate an instrument of up to 5 mm diameter. The second, third and fourth instrument insertion devices 202, 203, 204 can all accommodate instruments of up to 5 mm diameter.

The three instrument insertion devices 702, 703, 704 extend in a direction towards a surgeon performing the procedure. The pull tab 79 can be used as a reference in this aspect, pointing away from the surgeon. The arrangement of the insertion devices is ergonomically efficient as the surgeon can readily manipulate tissue retraction and camera instruments inserted through the devices 702, 703, 704. The larger device 704 points away from the surgeon as it may be used only occasionally during the procedure, for example in removing dissected tissue. In this way the available space is optimised.

The device comprises a tab 729 which can be used for mounting and demouting the instrument access device from the proximal assembly

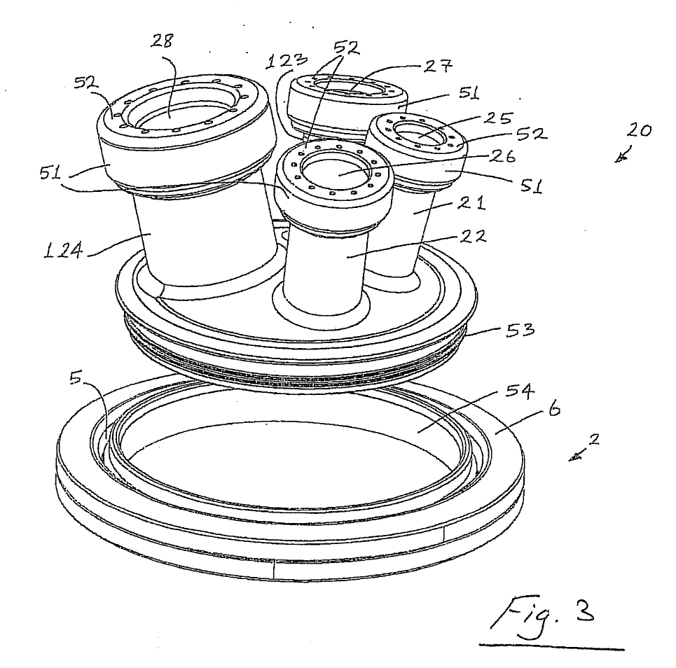

Referring to FIGS. 5 to 29 there is illustrated a method of performing a surgical procedure according to the invention. In this case the surgical procedure performed is a laparoscopic cholecystectomy procedure.

A number of medical devices may be employed to perform the procedure for example a scalpel 201, an introducer device 202, an instrument access device 203, an insufflator 204, a camera device 205, and various surgical instruments 206.

In use, the introducer device 202 and the instrument access device 203 are supplied in a pack 207. The pack 207 is opened (FIG. 5), and the introducer device 202 and the instrument access device 203 are removed from the pack 207 (FIG. 6).

FIG. 6 illustrates the peel off lid 208, the triport device 203, the injector introducer 202, and the plastic blister pack tray 207 which is a sterile pack. In FIG. 6 the user peels open the tray lid 208. FIG. 7 illustrates the injector introducer 202, the thumbswitch 209, the blunt dissecting tip 210, the distal ring 211, the sleeve 212, the removal ribbon 213, the removal ring 214, the insufflation line 215, the 5 mm ports 216, the 12 mm port 217, and the outer proximal ring 218. In FIG. 7 the user removes the introducer 202, and the triport 203.

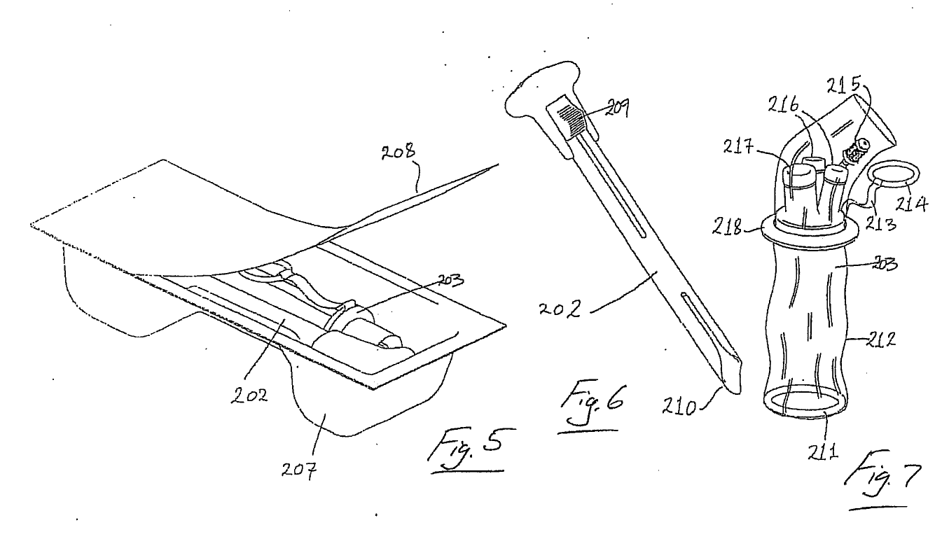

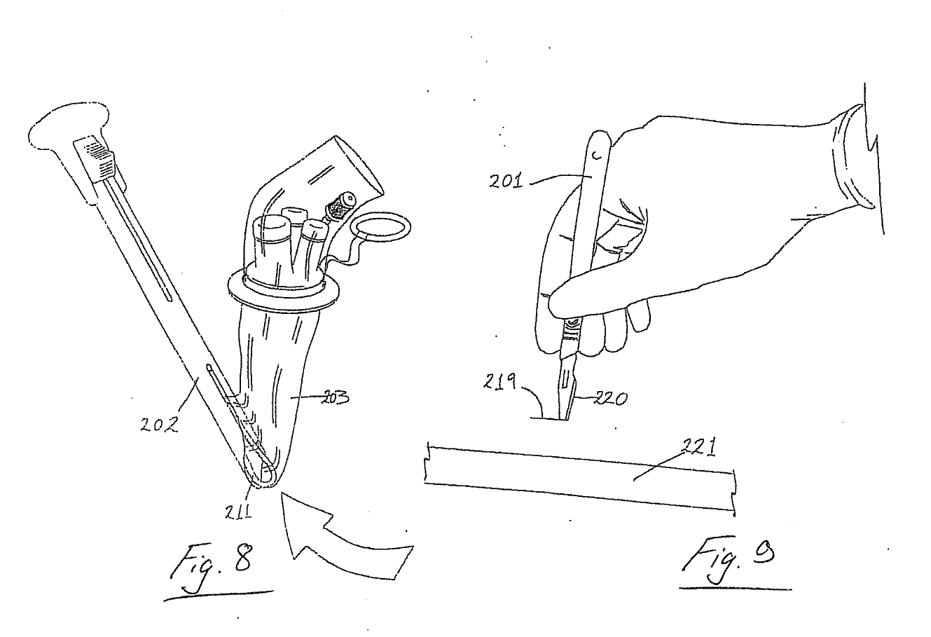

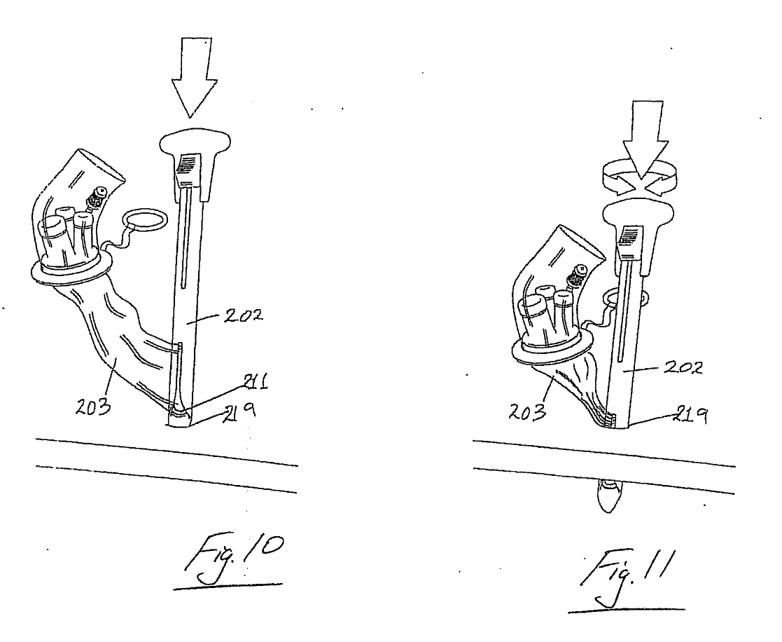

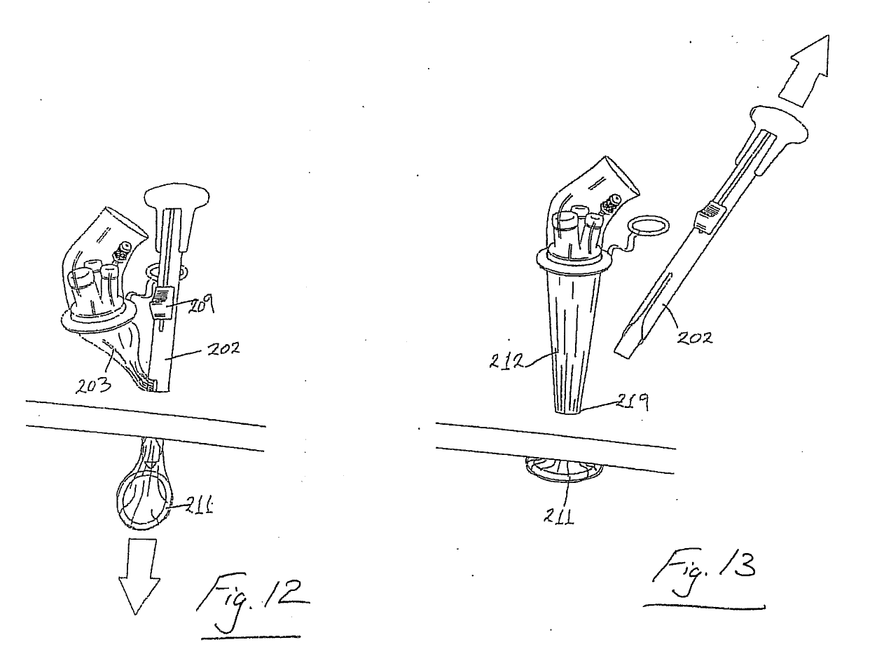

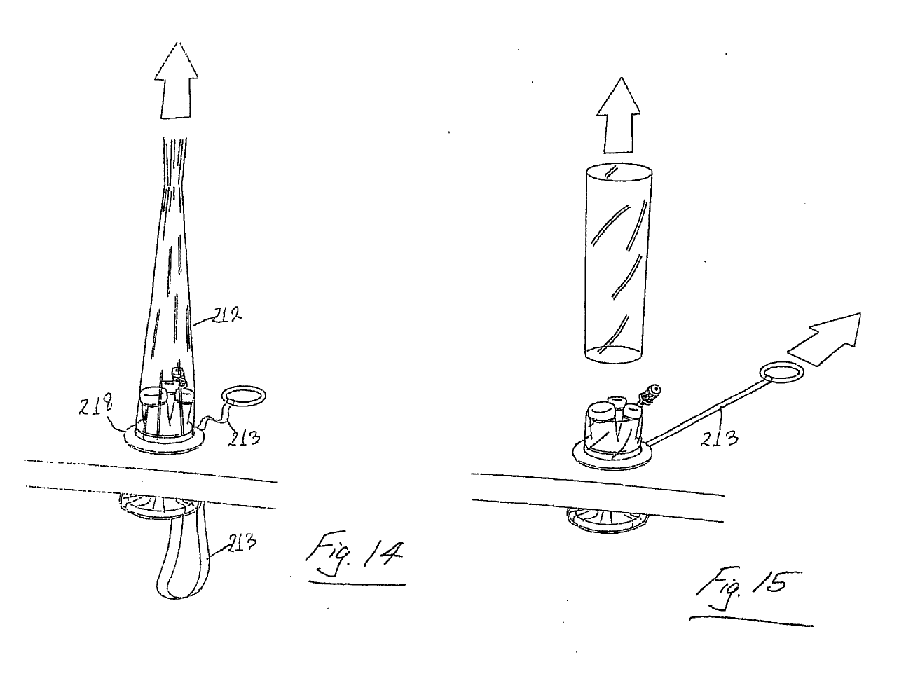

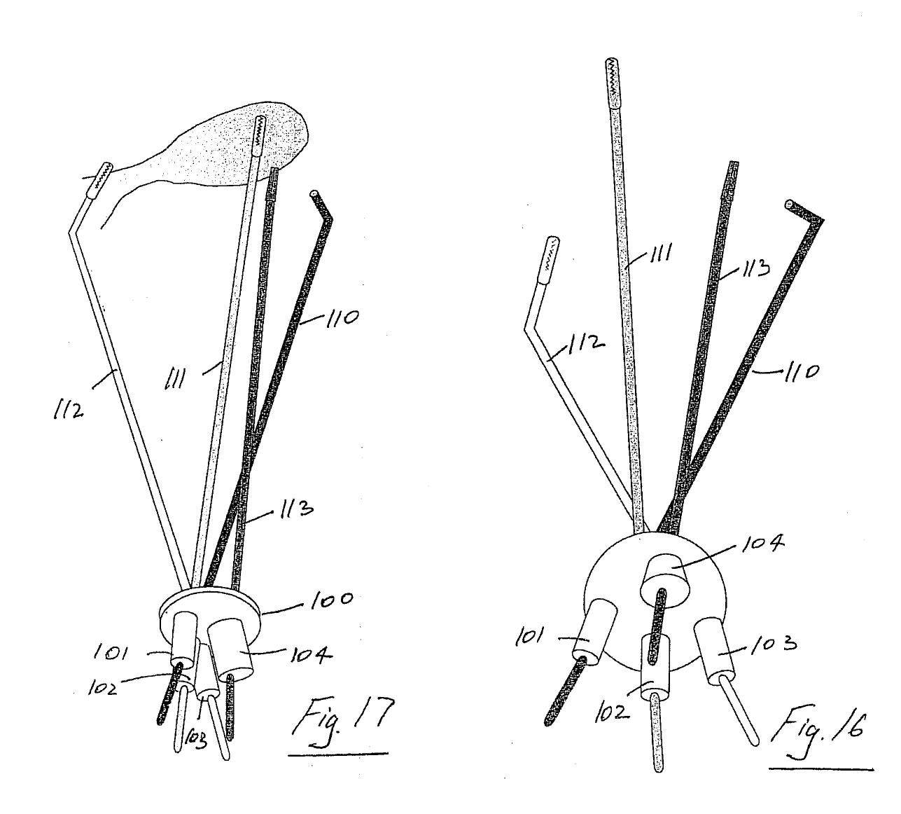

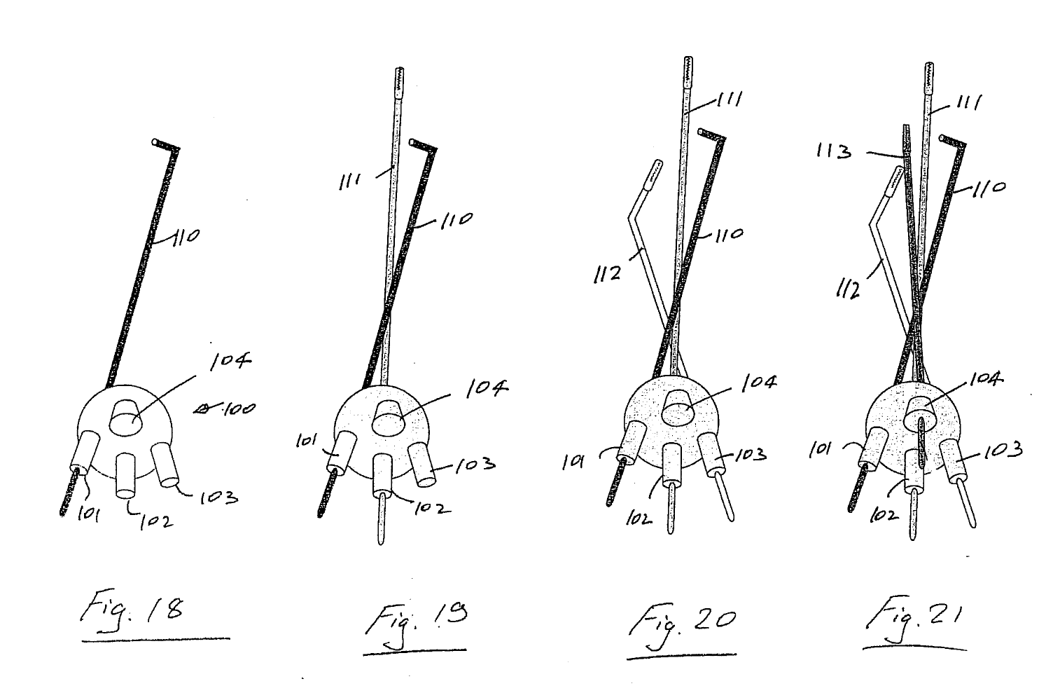

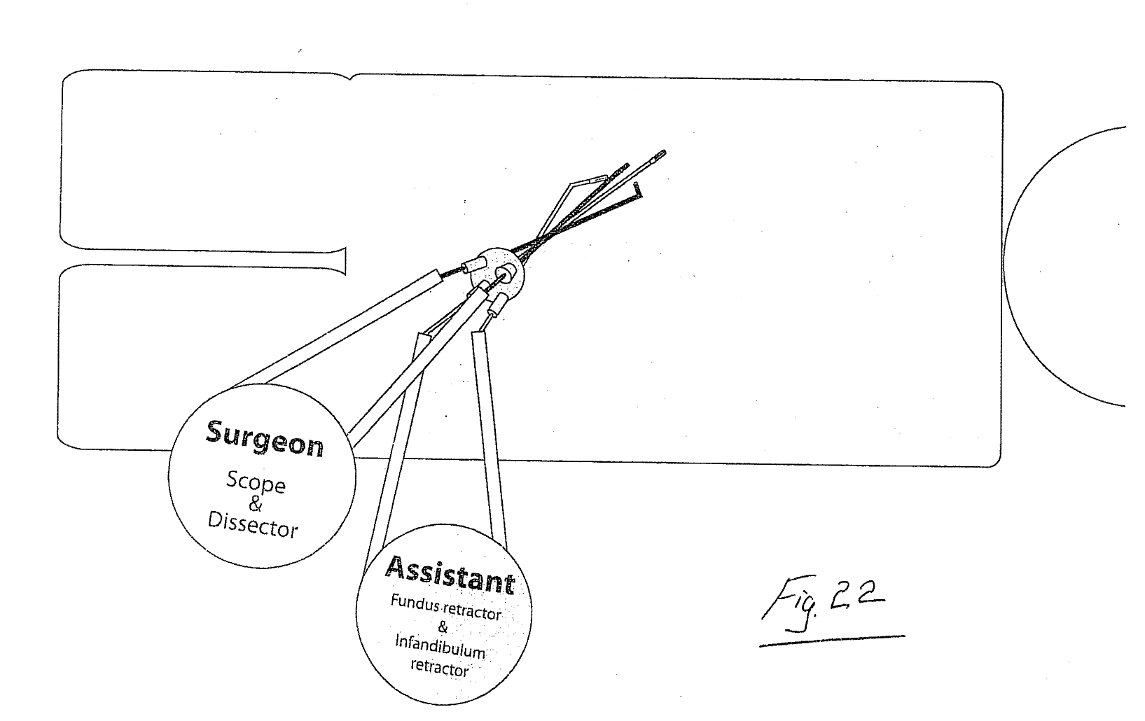

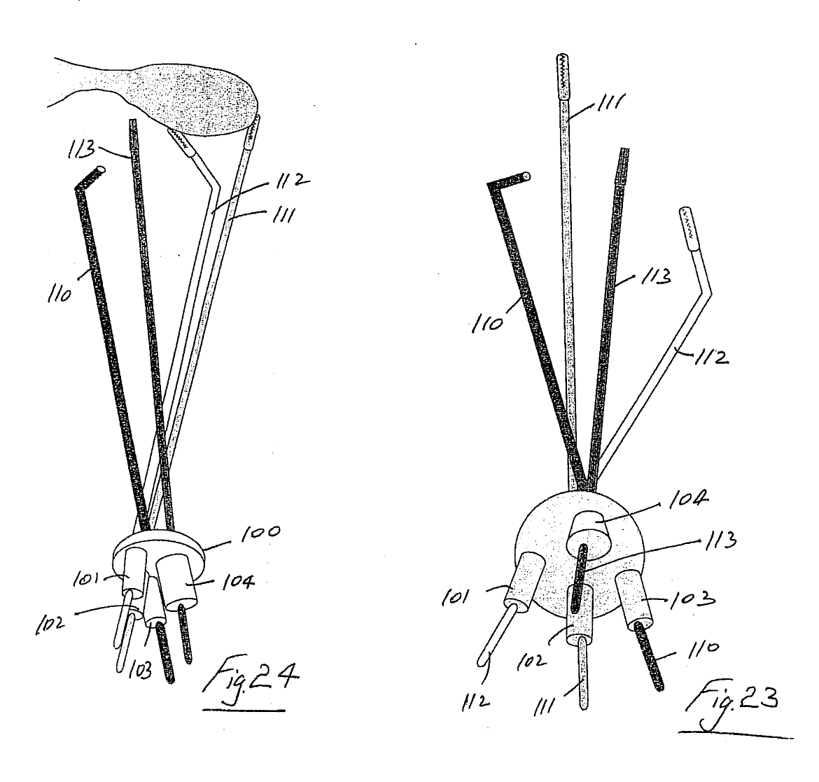

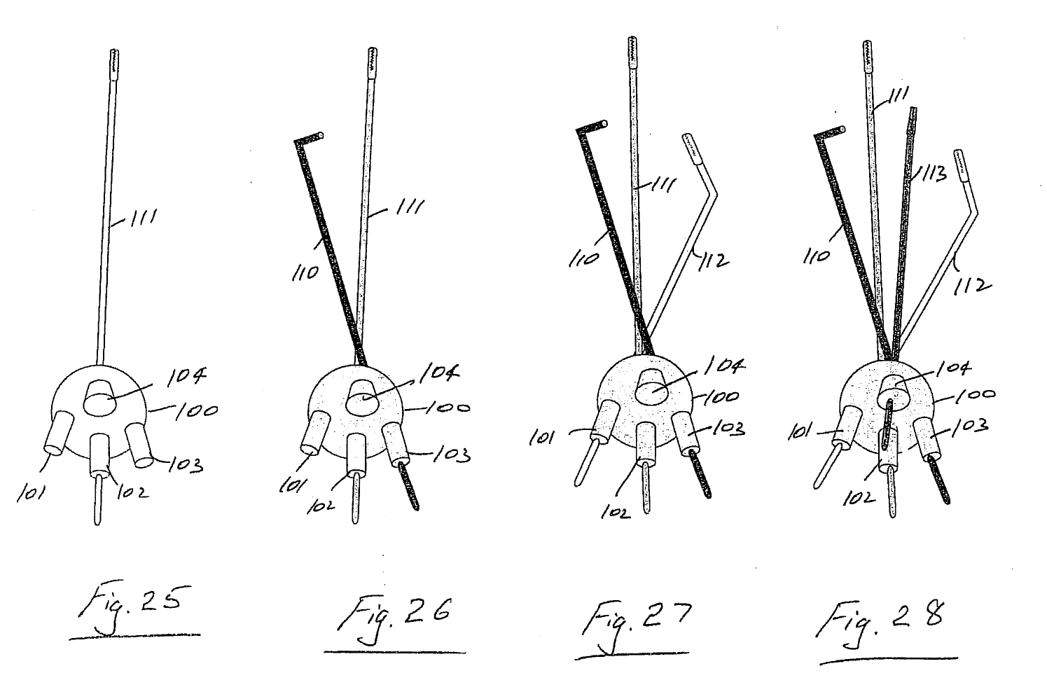

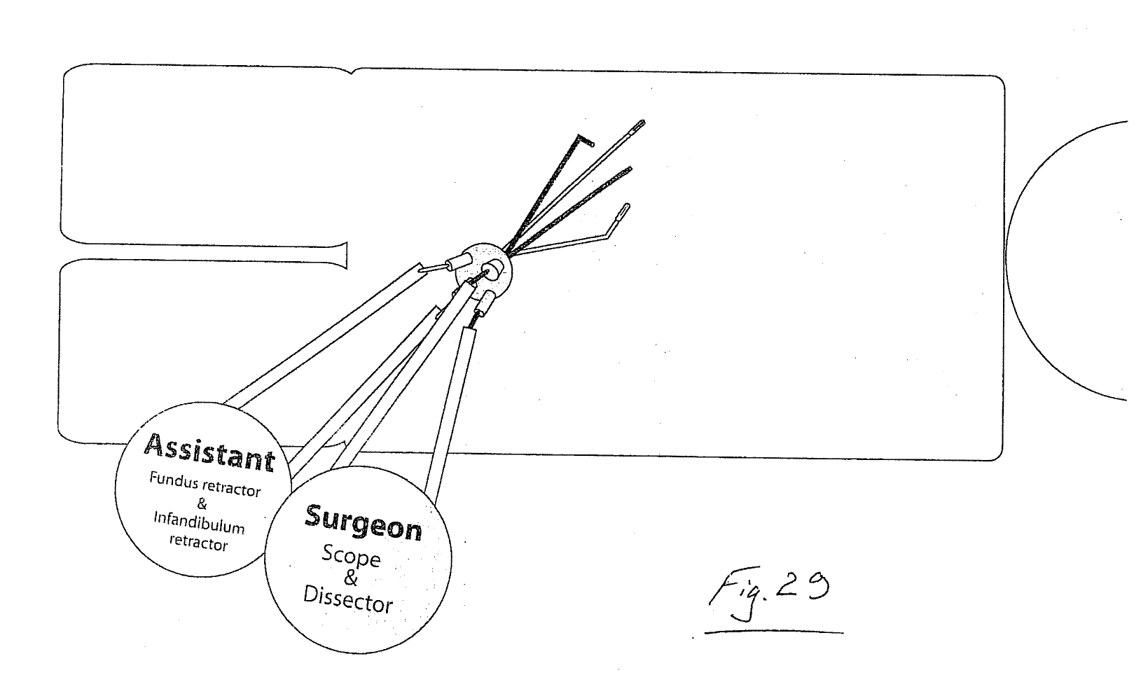

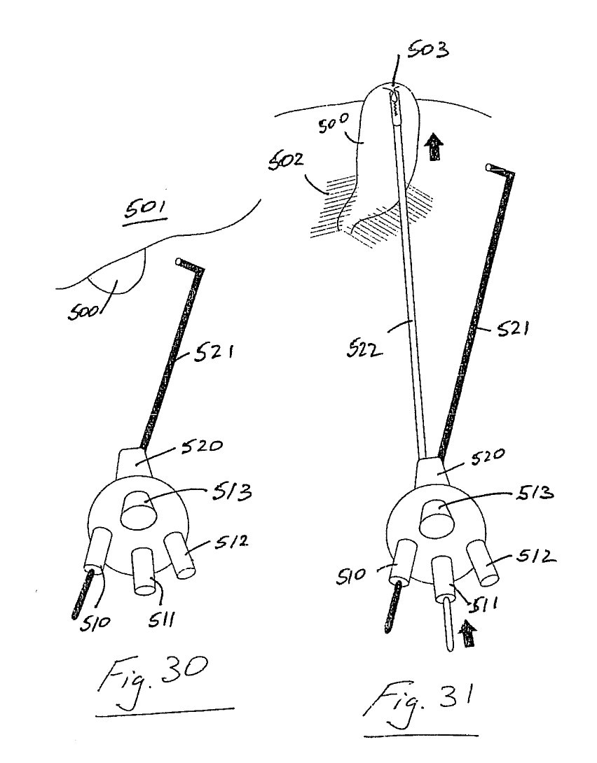

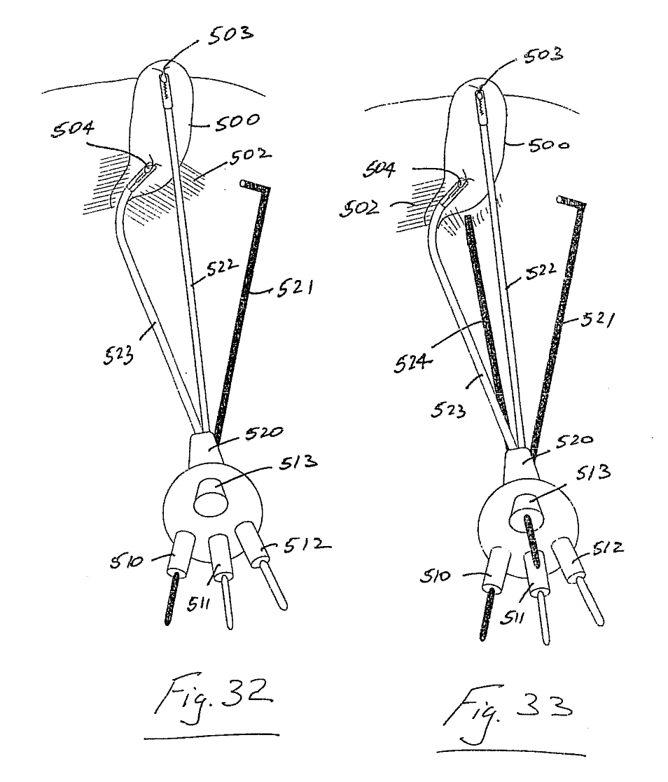

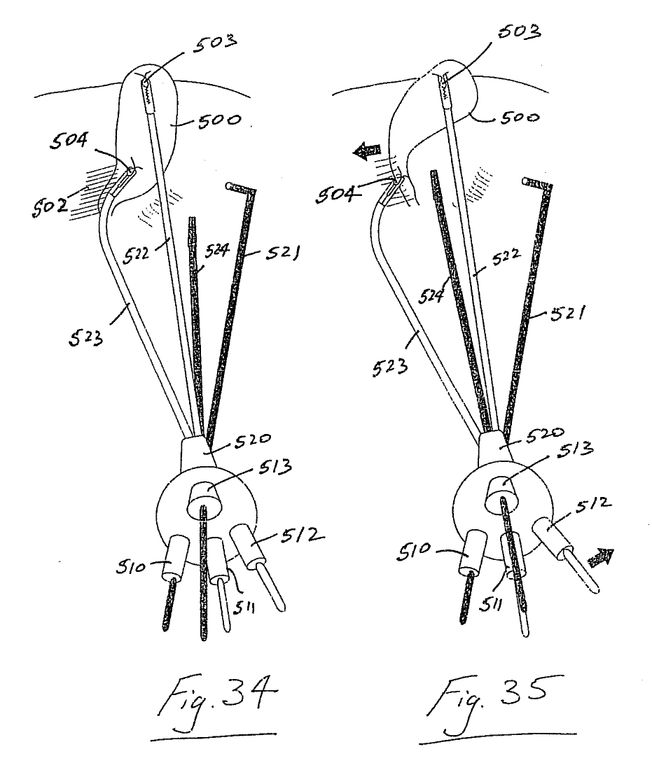

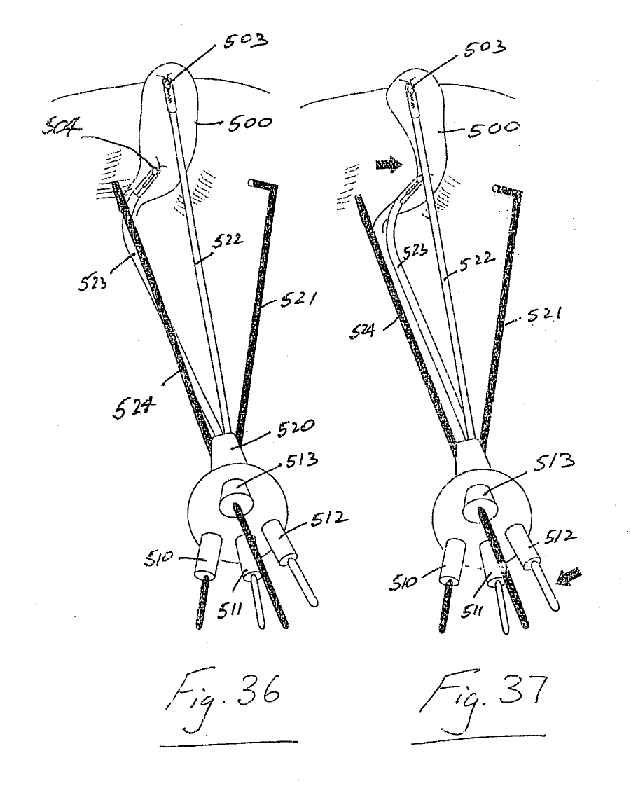

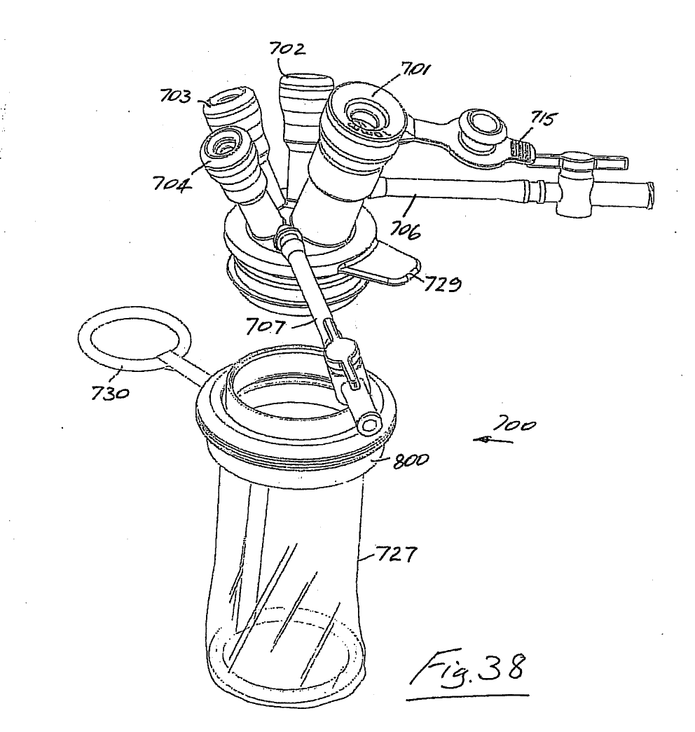

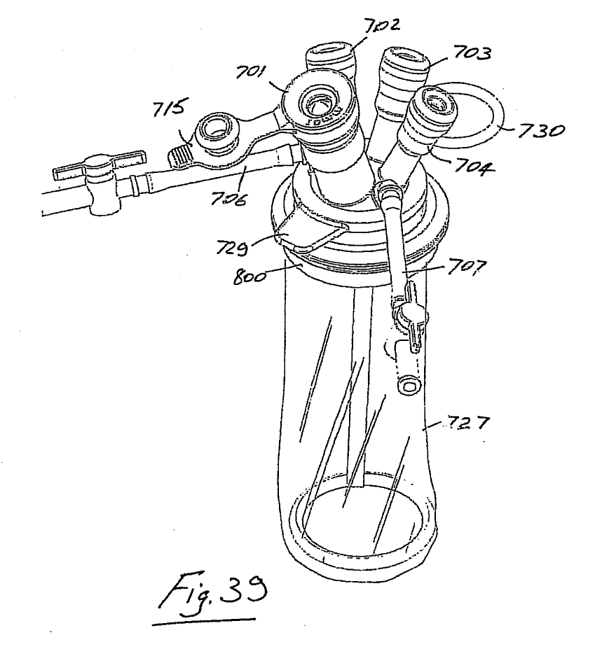

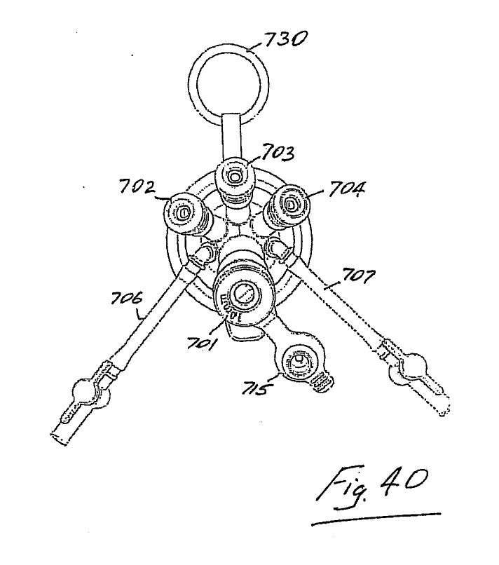

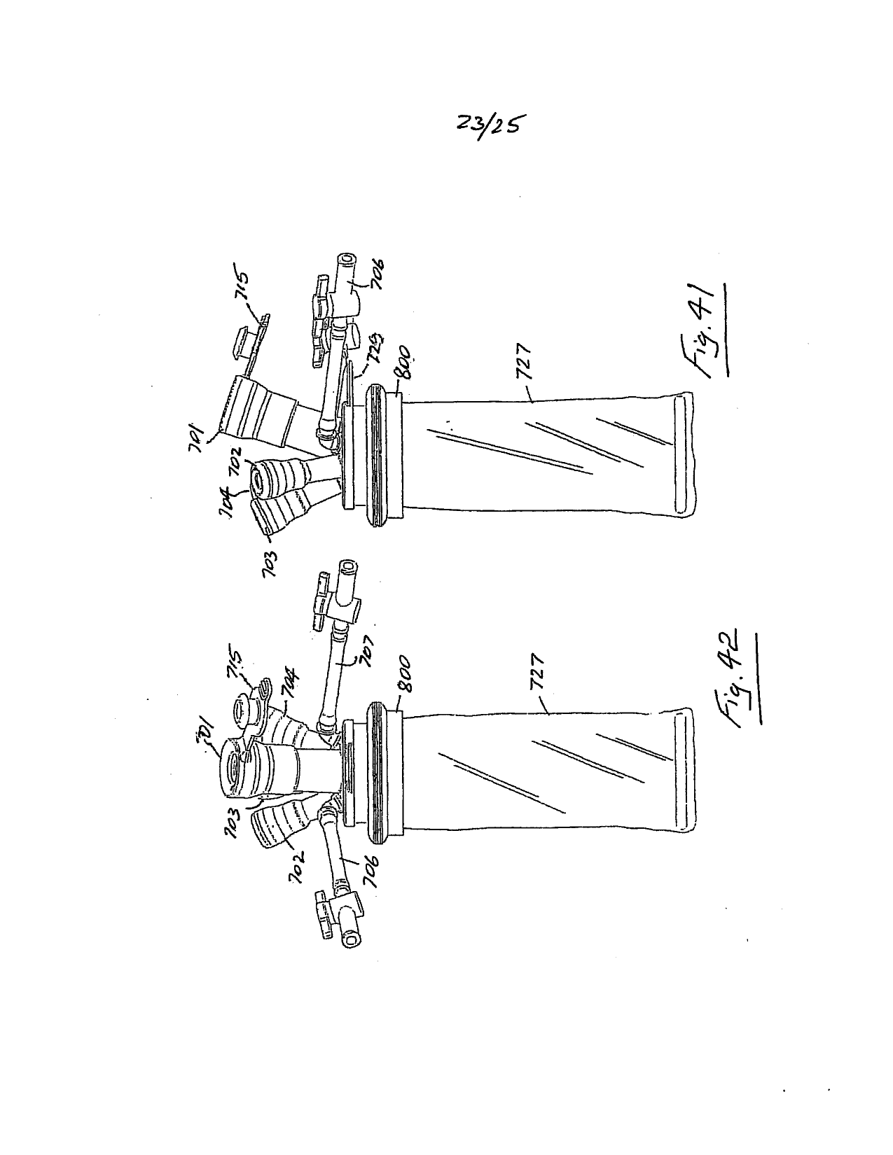

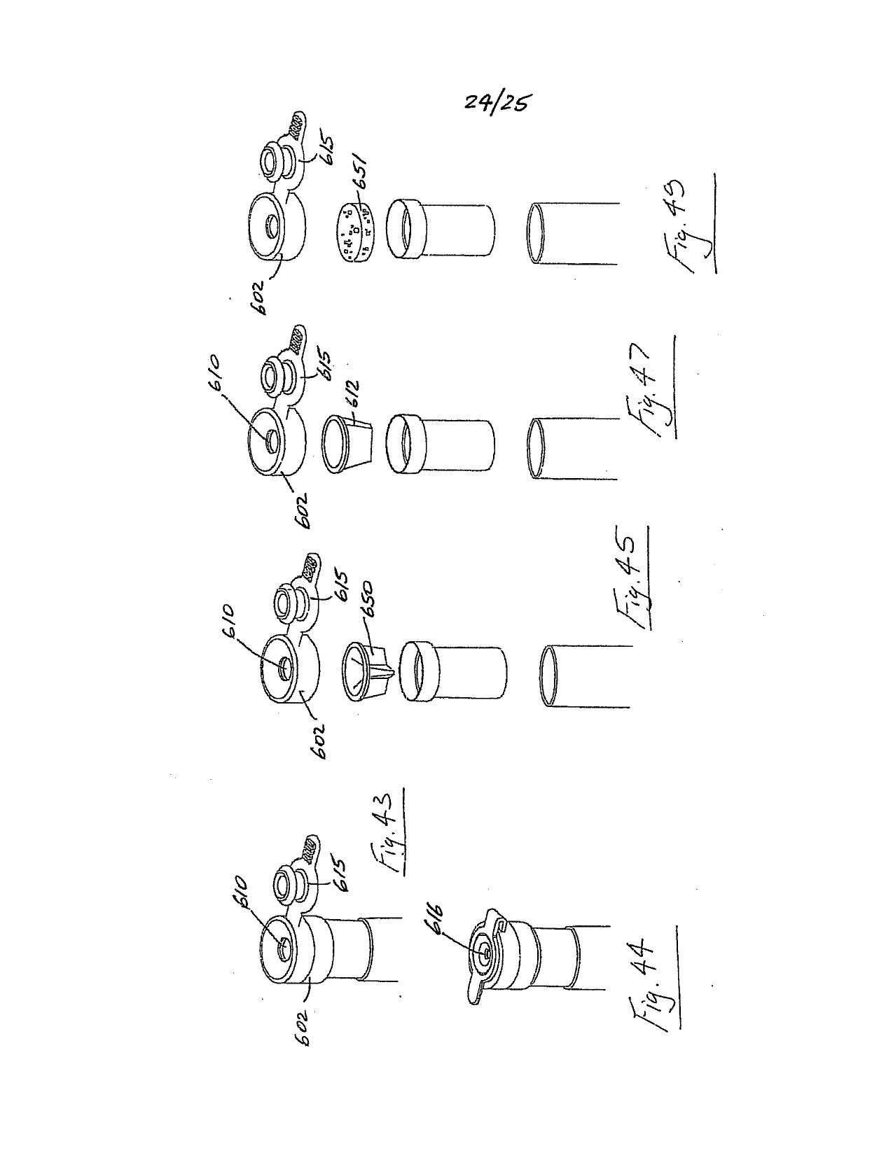

The distal ring 211 of the instrument access device 203 is inserted into the introducer device 202 (FIG. 8), and the scalpel 201 is used to create a wound opening 219 (FIG. 9).

In FIG. 8 the user inserts the distal ring 211 into the end of the injector introducer 202. FIG. 9 illustrates the scalpel 201, the blade 220, and the abdominal wall 221. In FIG. 9 the surgeon creates either a 15-20 mm skin incision 219 through the skin and the fascia layers but not through the peritoneum, or cuts all the way through to the abdominal cavity with a Hasson cut-down incision 219.

The introducer device 202 is inserted through the wound opening 219 until the distal ring 211 of the instrument access device 203 is within the wound interior (FIGS. 10 and 11).

In FIG. 10 the tip of the injector introducer 202 is placed in the skin incision 219 or the Hasson cut-down incision 219. In the case of the skin incision, downward pressure and axial rotation of the injector introducer 202 cause the blunt dissecting tip 210 to burrow through the peritoneum to the abdomen (FIG. 11). This may take place while the abdomen is insufflated. In the case of the Hasson cut-down incision, the injector 202 is easily passed through the pre-made incision (FIG. 11).

The thumbstitch 209 of the introducer device 202 is depressed to eject the distal ring 211 of the instrument access device 203 into the wound interior (FIG. 12), and the introducer device 202 is removed from the wound opening 219 (FIG. 13).

In FIG. 12 the thumbswitch 209 is pressed downwards to eject the distal ring 211. In FIG. 13 the injector introducer 202 is removed from the incision 219 leaving the distal ring 211 in the abdomen. The sleeve 212 is pulled upwards to engage the distal ring 211 with the underside of the abdominal wall.

The sleeve 212 of the instrument access device 203 is pulled proximally and the outer proximal ring 218 is pushed distally to retract laterally the sides of the wound opening 219 (FIG. 14). The excess sleeve material is cut-away, and the removal ribbon 213 is pulled proximally to remove any excess ribbon from the wound interior (FIG. 15).

In FIG. 14, the user keeps upward tension on the sleeve 212, and the outer proximal ring 218 is pushed down until sufficient retraction is achieved. In FIG. 15 the removal ribbon 213 is gently pulled to remove slackness from inside the abdomen. The excess sleeve 212 is cut and removed.

An insufflator is connected to the insufflation line to insufflate the abdomen and one or more instruments may be inserted through the ports.

Referring to FIGS. 16 to 22 there are illustrated various steps in a method utilising a multiport access device to carry out a surgical procedure. In this case the surgical procedure is a laparascopic cholecystectomy procedure.

A multiport access device 100 is used in the procedure. The device comprises four access ports 101, 102, 103, 104 which are used with various camera/scopes and instruments 110, 111, 112, 113.

After anesthetizing the skin with anesthetic a small incision is performed hidden inside the umbilicus to expose the fascia. A 1.5 cm vertical fascial defect is created and the abdomen is entered using a standard Hasson technique. The access device is deployed as described above. The abdomen is insuflated and the patient is placed in a reverse Trendelenburg position. For performance of the procedure the access port 100 is positioned to allow for the operating surgeon to control a camera 110 and a working instrument, while an assistant may hold stationary instruments. As illustrated in FIG. 22 the surgeon and assistant both stand on the patients left side with the assistant in front of the operating surgeon. The access device 100 is configured in the shape of a smile with three 5 mm ports 101, 102, 103 at the running inferiorly along the bottom and a larger 10 mm port 104 positioned in the midline at the top. The external arrangement of the operating devices is as follows. A camera 110 is placed in port 101 on the far left. An instrument 111 for grasping and elevating the gall bladder at the fundus is placed in port 102. A curved instrument 112 used for grasping and retraction of the infundibulum is placed in port 103. Functional instruments 113 used for dissection and clipping are placed through the 10 mm port 104. The internal arrangement of the operating devices is as follows. The camera 110 is positioned to be looking from the medial aspect toward the right side. Above the camera 110 in a linear plane is the fundal retraction instrument 111. Below the camera 110 is the dissection instruments 113 and finally in the linear plane will be the curve instrument 112 that is directed to the right side of the abdomen wall and whose natural curve is pointed back towards the gall bladder. There is also an approximate 10° angulation upward toward the infundibulum of the gall bladder. An identifying stripe on the instrument will allow the surgeon to be aware of the proper oreintation of the instrument. This positioning allows for lateral retraction of the infundibulum. The space created by positioning this instrument at a small angulation will allow for passage of the dissecting instruments in and out of the abdomen.

Referring to FIGS. 23 to 29, for dissection of the posterior aspect of the gall bladder, the camera 110 and curved instrument 112 retracting the infundibulum are reversed. The camera 110 is placed in the port 103 and directed toward the right side of the abdomen looking back medially. The curved instrument 112 is placed in port 101 and directed toward the left with the natural curve coming back to the right to grasp the infundibulum for medial retraction.

In FIG. 18 a scope 110 is shown placed through the lower left part 101 (30° or deflectable tip). In FIG. 19 a fundus retractor 111 is placed through the lower centre port 102 and under the scope 110. Referring to FIG. 20 the infandibulum curved retractor 112 is placed through the lower right port 103 and under the instruments 110, 111. As illustrated in FIG. 21 the dissector 113 is then placed through the top port 104 and above the other instruments 110, 111, 112.

In switching to expose the left side the fundus retractor 111 remains in place (FIG. 25). A scope 110 is placed through the lower right port 103 and above the fundus retractor 111 (FIG. 26). The infandibulum retractor 112 is then placed through the lower left port, below the fundus retractor 111 and the scope 110 (FIG. 27). The dissector 113 is then placed through the top port 104 and above all of the other instruments 101, 102, 103.

The method allows the gall bladder to be removed in a highly efficient manner using just one access port.

The dissected gall bladder can be removed through the port 104 or, if necessary the access device 100 can be released and the gall bladder removed through the incision.

Referring to FIGS. 30 to 37 various alternative steps in a surgical procedure involving a gall bladder are illustrated. In these drawings the gall bladder is assigned the reference numeral 500, the liver 501, the mesentery 502, the fundus 503 and the infundibulum 504.

The instrument access device many be similar to those described herein and in this case comprises a first instrument access port 510, a second instrument access port 511, a third instrument access port 512, and a further instrument access port 513. The device also has a removal tab 520. The procedure involves the use of a first instrument 521, a second instrument 522, a third instrument 523, and a fourth instrument 524 which are inserted into the access ports 510, 511, 512, 513 respectively.

Referring to FIG. 30 the access device is inserted with the removal tab 520 pointed towards the gall bladder 500. An instrument 521 with a 30 degree or deflectable tip scope is inserted through the first access port 510.

A grasper instrument 522 is then inserted through the second port 511 (FIG. 31). The fundus 503 of the gall bladder 500 is grasped and pushed back to expose the gall bladder 500. The instrument 522 can be placed above or below the scope 521.

A curved grasper instrument 523 is inserted through the third port 512 underneath the other instruments 521, 522 (FIG. 32). This grasper 523 grasps the infundibulum 504 of the gall bladder 500.

A dissector instrument 524 is then inserted through the port 513 towards the mesentery 502 (FIG. 33). As illustrated in FIG. 34 the mesentery 502 is then dissected from the right hand side of the gall bladder 500. Because the mesentery 502 is now dissected from the right hand side, the gall bladder 500 can be readily retracted to the left by moving the curved grasper 523 to the right as illustrated in FIG. 35.

Referring to FIG. 36 the mesentery 502 is now dissected from the left side of the gall bladder 500.

As the mesentery 502 is now dissected from both the right and left sides of the gall bladder 500, the gall bladder 500 can be readily retracted to the right by moving the curved grasper 523 to the left (FIG. 37).

The method allows the gall bladder to be removed in a highly efficient manner using just one access port. The dissected gall bladder can be removed through the port 513 or, if necessary, the access device can be released from the proximal assembly optionally by using the tab 520 and the gall bladder removed through the incision.

Various features of the invention are described and illustrated. It will be appreciated that at least some of the features described in relation to one embodiment may be used not only in the embodiment specifically described but also in other appropriate embodiments.

The invention is not limited to the embodiments hereinbefore described, with reference to the accompanying drawings, which may be varied in construction and detail.

1. A method for performing a cholecystectomy procedure comprising the steps of:
providing an access device having a plurality of ports for reception of instruments;
making an incision in the abdomen of a patient;
mounting the access device to provide access through the incision;
inserting a first grasper device through a first port of the access device;
inserting a second grasper device through a second port of the access device;
inserting a dissector through a third port of the access device; and
detaching at least a first portion of the gall bladder from surrounding tissue.

2. A method as claimed in claim 1 comprising:
moving the partially dissected gall bladder using at least one of the grasper devices, and
detatching another portion of the gall bladder from surrounding tissue.

3. A method as claimed in claim 1 comprising grasping the fundus of the gall bladder using the first grasper device.

4. A method as claimed in claim 1 comprising grasping the infundibulum of the gall bladder using the second grasper device.

5. A method as claimed in claim 1 comprising the step of inserting a camera through a fourth port of the access device in advance of inserting one or both of the grasper devices.

6. A method for performing a cholecystectomy procedure comprising the steps of:
providing an access device having a plurality of ports for reception of instruments;
making an incision in the abdomen of a patient;
mounting the access device to provide access through the incision;
inserting a first grasper device through a first port of the access device;
inserting a second grasper device through a second port of the access device;
inserting a dissector through a third port of the access device;
grasping the fundus of the gall bladder using the first grasper device;
grasping the infundibulum of the gall bladder using the second grasper device;
detaching at least a first portion of the gall bladder from surrounding tissue using the dissector;
moving the partially detached gall bladder using at least one of the graspers to expose another portion of tissue surrounding the gall bladder; and
detaching the further portion of the gall bladder from surrounding tissue using the dissector.

7. A method as claimed in claim 1 comprising:—
removing some of the instruments from the ports;
inserting instruments through the ports, and dissecting another portion of the gall bladder.

8. A method as claimed in claim 1 wherein the first grasper device comprises a fundus grasper device.

9. A method as claimed in claim 1 wherein the second grasper device comprises an infandibulum grasper.

10. A method as claimed in claim 9 wherein the infandibulum grasper has a curved shaft portion.

11. A method as claimed in claim 1 comprising holding the grasper devices separate from the dissector.

12. A method as claimed in claim 11 wherein the retractor devices are held by a surgical assistant.

13. A method as claimed in claim 1 wherein the method comprises the step of creating the wound opening.

14. A method as claimed in claim 13 wherein the wound opening is created by creating a skin incision, and subsequently forcing tissue apart.

15. A method as claimed in claim 13 wherein the wound opening is created using a Hasson cut-down incision.

16. A method as claimed in claim 1 wherein the method comprises the step of inserting an instrument access device at least partially through the wound opening.

17. A method as claimed in claim 16 wherein the instrument access device is inserted at least partially through the wound opening using an introducer device.

18. A method as claimed in claim 17 wherein the method comprises the step of inserting at least part of the instrument access device into the introducer device.

19. A method as claimed in claim 18 wherein the method comprises the step of inserting the introducer device at least partially through the wound opening.

20. A method as claimed in claim 19 wherein the method comprises the step of ejecting at least part of the instrument access device from the introducer device within the wound interior.

21. A method as claimed in claim 20 wherein the method comprises the step of removing the introducer device from the wound opening.

22. A method as claimed in claim 1 wherein the method comprises the step of retracting the wound opening.

23. A method as claimed in claim 1 wherein the method comprises the step of insufflating the wound interior.

24. A method as claimed in claim 1 wherein the one or more body parts are removed through one or more ports of the instrument access device.

25. A method as claimed in claim 22 wherein the method comprises the step of detaching the access device from a retractor member of the instrument access device.

26. A method as claimed in claim 25 wherein the one or more body parts are removed through the retractor member.
